(19) United States
(12) Patent Application Publication
(10) Pub. No.: US 2026/0102462 A1
(43) Pub. Date: Apr. 16, 2026

(54) METHOD OF PREVENTING AND/OR TREATING CANCER BY SMALL- MOLECULE PROTEIN

(71) Applicant: National Chung Hsing University, Taichung City (TW)

(72) Inventors: Hui-Chih HUNG, Taichung City (TW); Ju-Yi HSIEH, Taichung City (TW)

(21) Appl. No.: 19/000,964

(22) Filed: Dec. 24, 2024

(30) Foreign Application Priority Data
Oct. 15, 2024 (TW) 113139160

(51) Int. Cl. A61K 38/17; A61P 35/00

(52) U.S. Cl. CPC A61K 38/1709; A61P 35/00

(57) ABSTRACT

A method of preventing and/or treating cancer by a small-molecule protein is disclosed. The small-molecule protein is a variant of an antizyme (AZ) protein and has the activity to degrade the ornithine decarboxylase (ODC) in cells. Therefore, administering an effective amount of the small-molecule protein to an individual can effectively inhibit the activity of the ODC in the individual's body, reduce the expression of polyamines in the individual's cells, and thereby produce the effect of treating cancer or slowing down cancer progression.

### REFERENCE TO AN ELECTRONIC SEQUENCE LISTING

The content of the electronic sequence listing (File Name: SequenceList.xml; Size: 13 kilo bytes; and Date of Creation: Dec. 10, 2024) is herein incorporated by reference in its entirety.

### BACKGROUND OF THE INVENTION

### 1. Technical Field

The present invention relates to a use of a small-molecule protein. More particularly, the invention relates to a method of preventing and/or treating cancer by a small-molecule protein.

### 2. Description of Related Art

Human ornithine decarboxylase (hereinafter referred to as ODC for short) is a rate-limiting enzyme participating in the initial stage of polyamine synthesis and is also a pyridoxal 5′-phosphate (PLP)-dependent enzyme. ODC can catalyze the decarboxylation of ornithine to produce diamine putrescine, thus promoting the synthesis of polyamines, and is therefore viewed as playing an important role in the polyamine biosynthesis path. A polyamine is an aliphatic compound that can be found in cells and has at least two amino groups, and is believed to be associated with such processes as embryonic development, the progression of cell cycles, differentiation, proliferation, autophagy, and apoptosis. Over-expression of polyamines in cells may lead to certain diseases such as cancer or other diseases related to excessive proliferation.

An antizyme (hereinafter referred to as AZ for short) can encourage rapid degradation of ODC and thereby control the expression of ODC in cells. As over-expression of ODC may facilitate the formation of cancer, ODC has been classified as a proto-oncogene, and the regulation of ODC with an AZ is critical to maintaining the expression of polyamines in cells. Other proteins that take part in polyamine control are the antizyme inhibitors (hereinafter referred to as AZIs for short). For example, an AZI binds to AZ1 to not only impair the interaction between AZ1 and ODC, but also serve as a negative regulator of AZ1, thus enhancing the biosynthesis of polyamines. AZIs, therefore, are considered oncoproteins.

### BRIEF SUMMARY OF THE INVENTION

The primary objective of the present invention is to provide a method that uses a small-molecule protein to treat and/or prevent cancer. More specifically, the method uses the small-molecule protein, or a composition containing the small-molecule protein, to regulate the expression of ODC and polyamines in an individual and thereby produce the effect of cancer treatment and/or prevention.

Another objective of the present invention is to provide a small-molecule protein for cancer treatment. The small-molecule protein, or a composition containing the small-molecule protein, can change the environment in cells and thereby regulate the expression of enzymes or other proteins in the cells, in order to affect cell growth or apoptosis.

To achieve the foregoing objectives, the present invention discloses a method of preventing and/or treating cancer by a small-molecule protein. The method essentially includes administering an effective amount of the small-molecule protein, or a composition containing the small-molecule protein, to an individual to inhibit polyamine expression in cells and enhance the activity of ODC degradation, thereby producing the effect of cancer treatment and/or prevention, wherein the small-molecule protein is a variant of an AZ protein.

In one embodiment of the present invention, the small-molecule protein is prepared by cutting off some of the amino acids in the N-terminal area of an AZ, and the amino acid sequence of the small-molecule protein includes SEQ ID No.: 1, SEQ ID No.: 2, or an amino acid sequence having at least 95% homology with SEQ ID No.: 1 or SEQ ID No.: 2.

More specifically, the amino acid sequence of the small-molecule protein consists of SEQ ID No.: 1 or SEQ ID No.: 2.

The aforesaid composition is a pharmaceutical that can be used to treat and/or prevent cancer, regulate the expression of polyamines in cells, or regulate the speed at which ODC is degraded in cells.

### BRIEF DESCRIPTION OF THE SEVERAL VIEWS OF THE DRAWINGS

FIG. 1 shows the results of analyzing the protein expression of AZ1₁₋₂₂₈ and AZ1₃₄₋₂₂₈ through a cycloheximide (CHX) chase assay.

FIG. 2 shows the results of analyzing the protein expression of AZ1₃₄₋₂₂₈ and AZ1₄₈₋₂₂₈ through a CHX chase assay.

FIG. 3 shows the results of analyzing the protein expression of AZ1₃₄₋₂₂₈ and AZ1₆₁₋₂₂₈ through a CHX chase assay.

FIG. 4 shows the results of analyzing the protein expression of AZ1₃₄₋₂₂₈ and AZ1₇₂₋₂₂₈ through a CHX chase assay.

FIG. 5 shows the results of analyzing the protein expression of AZ1₃₄₋₂₂₈ and AZ1₈₃₋₂₂₈ through a CHX chase assay.

FIG. 6 shows the results of analyzing the protein expression of AZ1₃₄₋₂₂₈ and AZ1₉₅₋₂₂₈ through a CHX chase assay.

FIG. 7 shows the results of analyzing the protein expression of AZ2₁₋₁₈₉ and AZ2₄₇₋₁₈₉ through a CHX chase assay.

FIG. 8 shows the results of analyzing the protein expression of AZ3 isoform-1 and AZ3 isoform-2 through a CHX chase assay.

FIG. 9 shows the results of analyzing, by the immunoblot method, the protein expression in HEK293T cells transfected with a plasmid with the ODC gene and in HEK293T cells transfected with a plasmid with the AZ1₃₄₋₂₂₈ and ODC genes.

FIG. 10 shows the results of analyzing, through a CHX chase assay, the ODC expression in HEK293T cells transfected with a plasmid with the ODC gene and in HEK293T cells transfected with a plasmid with the AZ1₃₄₋₂₂₈ and ODC genes.

FIG. 11 shows the results of analyzing, by the immunoblot method, the protein expression in HEK293T cells transfected with a plasmid with the AZ1₃₄₋₂₂₈ and ODC genes and in HEK293T cells transfected with a plasmid with the AZ1₄₈₋₂₂₈ and ODC genes.

FIG. 12 shows the results of analyzing, through a CHX chase assay, the ODC expression in HEK293T cells transfected with a plasmid with the AZ1₃₄₋₂₂₈ and ODC genes and in HEK293T cells transfected with a plasmid with the AZ1₄₈₋₂₂₈ and ODC genes.

FIG. 13 shows the results of analyzing, by the immunoblot method, the protein expression in HEK293T cells transfected with a plasmid with the AZ1₃₄₋₂₂₈ and ODC genes, in HEK293T cells transfected with a plasmid with the AZ1₆₁₋₂₂₈ and ODC genes, and in HEK293T cells transfected with a plasmid with the AZ1₇₂₋₂₂₈ and ODC genes.

FIG. 14 shows the results of analyzing, through a CHX chase assay, the ODC expression in HEK293T cells transfected with a plasmid with the AZ1₃₄₋₂₂₈ and ODC genes, in HEK293T cells transfected with a plasmid with the AZ1₆₁₋₂₂₈ and ODC genes, and in HEK293T cells transfected with a plasmid with the AZ1₇₂₋₂₂₈ and ODC genes.

FIG. 15 shows the results of analyzing, by the immunoblot method, the protein expression in HEK293T cells transfected with a plasmid with the AZ1₃₄₋₂₂₈ and ODC genes and in HEK293T cells transfected with a plasmid with the AZ1₈₃₋₂₂₈ and ODC genes.

FIG. 16 shows the results of analyzing, through a CHX chase assay, the ODC expression in HEK293T cells transfected with a plasmid with the AZ1₃₄₋₂₂₈ and ODC genes and in HEK293T cells transfected with a plasmid with the AZ1₈₃₋₂₂₈ and ODC genes.

FIG. 17 shows the results of analyzing, by the immunoblot method, the protein expression in HEK293T cells transfected with a plasmid with the AZ1₃₄₋₂₂₈ and ODC genes and in HEK293T cells transfected with a plasmid with the AZ1₉₅₋₂₂₈ and ODC genes.

FIG. 18 shows the results of analyzing, through a CHX chase assay, the ODC expression in HEK293T cells transfected with a plasmid with the AZ1₃₄₋₂₂₈ and ODC genes and in HEK293T cells transfected with a plasmid with the AZ1₉₅₋₂₂₈ and ODC genes.

FIG. 19 shows the test results of the polyamine content of each group of cells in example 5.

### DETAILED DESCRIPTION OF THE INVENTION

The present invention discloses a method of preventing and/or treating cancer by a small-molecule protein. The method includes administering an effective amount of the small-molecule protein, or a composition containing the small-molecule protein, to an individual to effectively inhibit the ODC activity in the individual's body, to reduce polyamine expression in the individual's cells, to prevent excessive cell proliferation, to block the genesis of cancer cells, and to thereby produce the effect of treating cancer or slowing down cancer progression. The small-molecule protein is a variant of an AZ protein and has the activity to degrade the ODC in cells.

In one embodiment of the present invention, the small-molecule protein is an AZ1 protein with a truncated N terminal, and the amino acid sequence of the small-molecule protein includes SEQ ID No.: 1, SEQ ID No.: 2, or an amino acid sequence that is at least 95% homologous with SEQ ID No.: 1 or SEQ ID No.: 2.

In one embodiment of the present invention in which the amino acid sequence of the small-molecule protein consists of SEQ ID No.: 1 or SEQ ID No.: 2, the small-molecule protein is highly stable in cells and has relatively high activity in inhibiting ODC and polyamine expression.

Table 1 shows the two major alternatives of the sequence of the small-molecule protein disclosed in the present invention.

TABLE 1
Two major alternatives of the sequence of the
small-molecule protein disclosed in
the present invention

| Name | Amino acid sequence | Sequence ID No. |
|---|---|---|
| AZ1₈₃₋₂₂₈ | MNSQRDHNLSANLFYSDDRLNVTEE | SEQ ID No.: 1 |
| | LTSNDKTRILNVQSRLTDAKRINWR | |
| | TVLSGGSLYIEIPGGALPEGSKDSF | |
| | AVLLEFAEEQLRADHVFICFHKNRE | |
| | DRAALLRTFSFLGFEIVRPGHPLVP | |
| | KRPDACFMAYTFERESSGEEEE | |
| AZ1₉₅₋₂₂₈ | MFYSDDRLNVTEELTSNDKTRILNV | SEQ ID No.: 2 |
| | QSRLTDAKRINWRTVLSGGSLYIEI | |
| | PGGALPEGSKDSFAVLLEFAEEQLR | |
| | ADHVFICFHKNREDRAALLRTFSFL | |
| | GFEIVRPGHPLVPKRPDACFMAYTF | |
| | ERESSGEEEE | |

In one embodiment of the present invention, the individual is a mammal such as a human, dog, or cat.

Unless otherwise stated, the scientific terms used in the present invention should be interpreted according to textbooks, literature, and/or general common knowledge in the field to which the invention pertains.

The term “AZ” is an abbreviation of “antizyme.” The synthesis of an AZ protein in cells is highly associated with the polyamine content in the cells; more specifically, the expression of polyamines is regulated by the AZ protein. The structural features of an AZ include two a helices and eight β sheets.

AZ isomers in mammals have been identified as AZ1, AZ2, and AZ3. Both AZ2 and AZ3 can interact with, and thereby inhibit the activity of, ODC and stop the polyamine uptake of cells.

Referring to Table 2, the human AZ1 protein, also known as the wild-type human AZ (abbreviated as AZ_WT) has 228 amino acids (GenBank: BAA23101.1) and can form AZ1₁₋₂₂₈ and AZ1₃₄₋₂₂₈, which start with the first and the 34^{th} amino acids respectively (Coffino P (2001a) Antizyme, a mediator of ubiquitin-independent proteasomal degradation. Biochimie 83:319-323; Matsufuji S, Matsufuji T, Miyazaki Y, Murakami Y, Atkins J F, Gesteland R F, Hayashi S (1995) Autoregulatory frameshifting in decoding mammalian ornithine decarboxylase antizyme. Cell 80:51-60; Rom E, Kahana C (1994) Polyamines regulate the expression of ornithine decarboxylase antizyme in vitro by inducing ribosomal frame-shifting. PNAS 91:3959-3963). The human AZ2 protein has 189 amino acids (UniProtKB/Swiss-Prot: 095190.1). The human AZ3 protein has two isoforms, namely AZ3 isoform-1, which has 235 amino acids (NCBI Sequence No.: NP_057262.2), and AZ3 isoform-2, which has 190 amino acids (NCBI Sequence No.: NP_001128411.1).

TABLE 2
Amino acid sequences of human AZ isomers

| AZ protein | Amino acid sequence | Sequence ID No. |
|---|---|---|
| AZ1 | MVKSSLQRILNSHCFAREKEGDKPSATIHA | SEQ ID |
| | SRTMPLLSLHSRGGSSSESSRVSLHCCSNP | No.: 3 |
| | GPGPRWCSDAPHPPLKIPGGRGNSQRDHNL | |
| | SANLFYSDDRLNVTEELTSNDKTRILNVQS | |
| | RLTDAKRINWRTVLSGGSLYIEIPGGALPE | |
| | GSKDSFAVLLEFAEEQLRADHVFICFHKNR | |
| | EDRAALLRTFSFLGFEIVRPGHPLVPKRPD | |
| | ACFMAYTFERESSGEEEE | |
| AZ2 | MINTQDSSILPLSNCPQLQCCRHIVPGPLW | SEQ ID |
| | CSDAPHPLSKIPGGRGGGRDPSLSALIYKD | No.: 4 |
| | EKLTVTQDLPVNDGKPHIVHFQYEVTEVKV | |
| | SSWDAVLSSQSLFVEIPDGLLADGSKEGLL | |
| | ALLEFAEEKMKVNYVFICFRKGREDRAPLL | |
| | KTFSFLGFEIVRPGHPCVPSRPDVMFMVYP | |
| | LDQNLSDED | |
| AZ3 | MPCKRCRPSVYSLSYIKRGKTRNYLYPIWS | SEQ ID |
| isoform-1 | PYAYYLYCYKYRITLREKMLPRCYKSITYK | No.: 5 |
| | EEEDLTLQPRSCLQCSESLVGLQEGKSTEQ | |
| | GNHDQLKELYSAGNLTVLATDPLLHQDPVQ | |
| | LDFHFRLTSQTSAHWHGLLCDRRLFLDIPY | |
| | QALDQGNRESLTATLEYVEEKTNVDSVFVN | |
| | FQNDRNDRGA LRAFSYMGFEVVRPDHPAL | |
| | PPLDNVIFMVYPLERDVGHLPSEPP | |
| AZ3 | MTVPWRPGKR RITYKEEEDL TLQPRSCLQC | SEQ ID |
| isoform-2 | SESLVGLQEG KSTEQGNHDQ LKELYSAGNL | No.: 6 |
| | TVLATDPLLH QDPVQLDFHF RLTSQTSAHW | |
| | HGLLCDRRLF LDIPYQALDQ GNRESLTATL | |
| | EYVEEKTNVD SVFVNFQNDR NDRGALLRAF | |
| | SYMGFEVVRP DHPALPPLDN VIFMVYPLER | |
| | DVGHLPSEPP | |

As used herein, the terms “AZ variant” or “AZ protein variant” refers to an AZ protein whose N-terminal area has been cut off partially, i.e., truncated, such as AZ1₃₄₋₂₂₈, AZ1₄₈₋₂₂₈, AZ1₆₁₋₂₂₈, AZ1₇₂₋₂₂₈, AZ1₈₃₋₂₂₈, and AZ1₉₅₋₂₂₈.

The term “AZI” is an abbreviation of “antizyme inhibitor,” which participates in polyamine regulation. AZIs bind to AZs to impair the interaction between AZs and ODC and serve as a negative regulator of AZs, thus promoting the biosynthesis of polyamines.

The term “ODC” is an abbreviation of “ornithine decarboxylase,” which consists of 461 amino acids and has a size of about 53 kDa. ODC works at the front end of polyamine biosynthesis and plays a rate-determining role in polyamine synthesis in mammals. Therefore, the ODC level or activity in the body of a mammal is associated with the expression of polyamines.

A “polyamine,” such as putrescine or spermine, is a group of positively charged small-molecules. The polyamines in a living organism can bind to DNAs, RNAs, or proteins, thereby affecting the folding, transcription, and translation of DNAs, and can therefore regulate the growth, apoptosis, differentiation, or gene expression of cells. When ODC is continuously expressed in cells, or when there is a huge amount of polyamines in cells, the speed of cell growth will increase, and the speed of apoptosis will lower such that cells proliferate abnormally and tend to develop cancer. That is to say, an increase in polyamine and ODC expression in cells increases the risk of cancer development.

The term “cycloheximide (CHX) chase assay” refers to applying a CHX reagent to a sample, measuring the expression of a target protein in the sample at different time points, and thereby estimating the degradation rate or half-life of the target protein.

The term “composition” refers to a substance that contains the small-molecule protein disclosed herein as the main active ingredient, that can be prepared in different dosage forms depending on such factors as the target users and the method of use, and that may be added with an excipient, a vehicle, and/or other ingredients that are pharmaceutically acceptable or are acceptable in the food industry. For example, the composition may be a pharmaceutical, a nutritional supplement, a food, or a preparation having a therapeutic effect.

The term “treating” refers to reversing, alleviating, or inhibiting the progression, or one or more symptoms, of the disease to which the term is applied or preventing the development or complications of the disease. The term “treatment” refers to a therapeutic action of “treating” as defined herein.

The technical features of the present invention and their effects are detailed below with reference to some examples and the accompanying drawings. The contents of the following examples serve only to explain or illustrate the appended claims and are not intended to be restrictive of the scope of the patent protection sought by the applicant.

Unless otherwise specified, all the proteins used in the following examples are derived from humans.

The proteins used in the following examples are designated by their respective English abbreviations or by names in the form of “English abbreviation x-Y,” in which X and Y are positive integers standing for the corresponding serial numbers of the amino acids in the amino acid sequence of the protein indicated by the English abbreviation, with X being the serial number of the amino acid in the “English abbreviation” protein that corresponds to the starting amino acid of the “English abbreviation x-Y” protein, and Y being the serial number of the amino acid in the “English abbreviation” protein that corresponds to the ending amino acid of the “English abbreviation x-y” protein. For example, “AZ1₃₄₋₂₂₈” refers to an N-terminal truncated AZ1 whose starting amino acid is the 34^{th} amino acid in the full-length sequence of AZ1 and whose ending amino acid is the 228^{th} amino acid in the full-length sequence of AZ1.

The cell lines and microorganisms used in the following examples are easily available to a person of ordinary skill in the art and therefore need not be deposited for patent purposes.

### Example 1: Protein Expression and Purification

Referring to Table 3, the gene of each protein was constructed in the corresponding vector to form a recombinant vector, which in turn was transferred into the corresponding microorganism in order for the protein to be expressed. As the expression of the proteins is known to be regulated by the lactose operon, isopropyl β-D-1-thiogalactopyranoside (IPTG) was added to induce protein expression, and the induction was allowed to take place at 25° C. for 20 hours. After purification, the proteins were verified by sodium dodecyl sulfate-polyacrylamide gel electrophoresis (SDS-PAGE), and the protein concentrations were verified by the Bradford method (Bradford M M (1976) A rapid and sensitive method for the quantitation of microgram quantities of protein utilizing the principle of protein-dye binding. Anal Biochem 72:248-254).

TABLE 3
Vectors and microorganisms used to prepare the
proteins for use in the present invention

| Protein name | Vector | Microorganism platform |
|---|---|---|
| AZ1 (AZ1₁₋₂₂₈) | pQE30 | E. coli JM109 |
| ODC | pQE30 | E. coli JM109 |
| Ubiquitin | pQE30 | E. coli JM109 |
| AZ2 (AZ2₁₋₁₈₉) | pET21b | E. coli BL21 (DE3) |
| AZ3 | pET28a | E. coli BL21 (DE3) |

### Example 2: Preparation of N-Terminal Truncated AZ Proteins

N-terminal truncation was performed on AZ proteins by inverse polymerase chain reaction (inverse PCR). Plasmid pcDNA3.1 containing the AZ1 or AZ2 gene was subjected to an amplification reaction in which the reverse primers listed in Table 4 and PfuUltra II fusion HS DNA polymerase were used, with more than 30 thermal cycles completed.

TABLE 4
Primers used in inverse PCR

| | | | Sequence |
|---|---|---|---|
| | Primer | Amino acid | ID |
| Protein | name | sequence | No. |
| AZ1 | Start from | ATGCCGCTCC | SEQ |
| | | TAAGCCTGCA | ID |
| | | | No.: |
| | | | 7 |
| | Start from | GAGAGTTCCA | SEQ |
| | | GGGTCTCCCT | ID |
| | | | No.: |
| | | | 8 |
| | Start from | GGTCCGGGGC | SEQ |
| | | CTCGGTG | ID |
| | | | No.: |
| | | | 9 |
| | Start from | CACCCACCCC | SEQ |
| | | TGAAGATCCC | ID |
| | | A | No.: |
| | | | 10 |
| | Start from | AATAGTCAGA | SEQ |
| | | GGGATCACAA | ID |
| | | TCTTTCA | No.: |
| | | | 11 |
| | Start from | TTCTACTCCG | SEQ |
| | | ATGATCGGCT | ID |
| | | GAAT | No.: |
| | | | 12 |
| AZ2 | Start from | GGCGGCAGGG | SEQ |
| | | ATCCTTCTCT | ID |
| | | | No.: |
| | | | 13 |

The PCR products were incubated at 37° C. for 30 minutes together with T4 polynucleotide kinase, and then digestion by restriction enzyme DpnI took place at 37° C. for 2 hours. Following that, the severed segments were connected with T4 ligase at room temperature for 3 hours and then transferred into E. coli XL-10 Gold® by way of transformation in order to be expressed, producing N-terminal truncated AZ proteins. After preliminary verification by colony PCR, the accuracy of the N-terminal truncated AZ proteins was verified by automatic sequencing.

The N-terminal truncated AZ protein variants obtained in this example include: AZ1₃₄₋₂₂₈, AZ1₄₈₋₂₂₈, AZ1₆₁₋₂₂₈, AZ1₇₂₋₂₂₈, AZ1₈₃₋₂₂₈, AZ1₉₅₋₂₂₈, and AZ2₄₇₋₁₈₉.

### Example 3: CHX Chase Assays of the N-Terminal Fragments of AZ Proteins

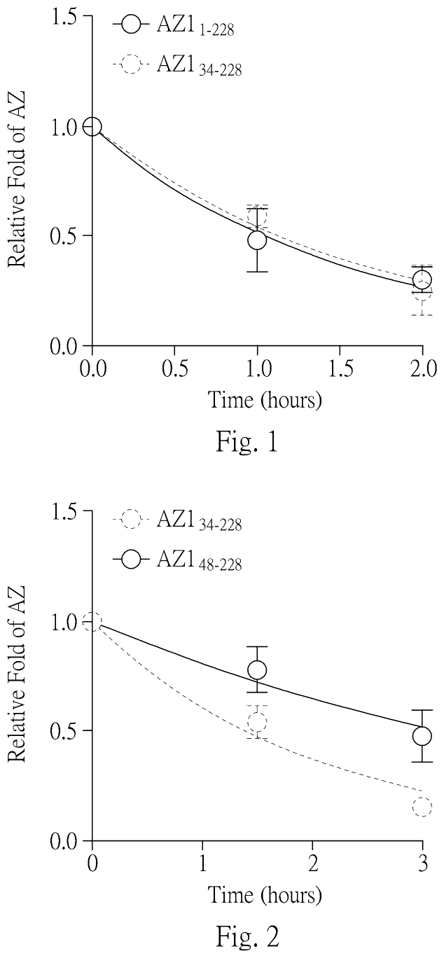

Genes each encoding one of AZ1₁₋₂₂₈, AZ1₃₄₋₂₂₈, AZ1₄₈₋₂₂₈, AZ1₆₁₋₂₂₈, AZ1₇₂₋₂₂₈, AZ1₈₃₋₂₂₈, and AZ1₉₅₋₂₂₈ were separately constructed in plasmid pcDNA3.1, and the resulting plasmids were transfected into HEK293T cells separately. The recombinant HEK293T cells were cultured for 24 hours, before CHX chase assays were performed to quantify and analyze the degradation rates of AZ1 and its variants. More specifically, after the cultivation, each group of recombinant HEK293T cells was added with the CHX reagent (cycloheximide) at 20 μg/mL to stop protein synthesis, and once the synthesis was stopped, the protein concentrations in the cells were measured at predetermined time points to analyze the half-lives of the proteins. The measurement and analysis results are shown in FIG. 1 to FIG. 6.

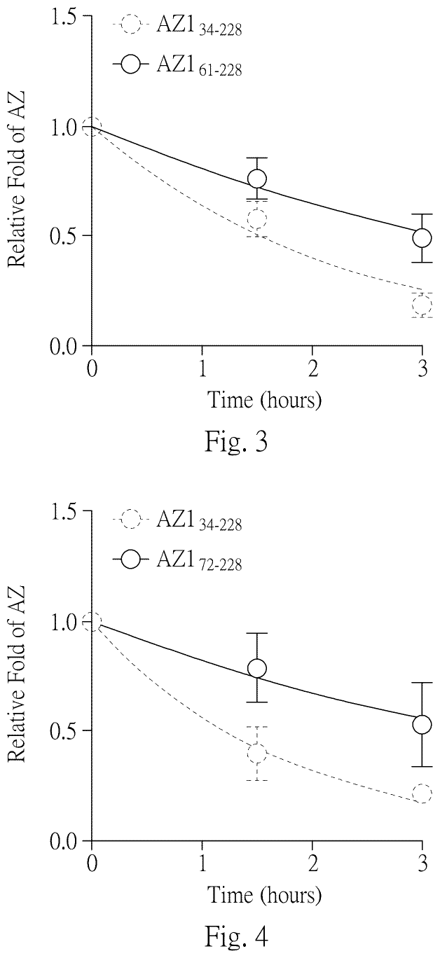

It can be known from the results in FIG. 1 that the AZ1₁₋₂₂₈ and AZ1₃₄₋₂₂₈ proteins have equivalent degradation rates, with the half-life of AZ1₁₋₂₂₈ being 1.04±0.22 hours, and the half-life of AZ1₃₄₋₂₂₈ being 1.13±0.20 hours. It can be known from the results in FIG. 2 that the half-life of AZ1₃₄₋₂₂₈ is 1.38±0.24 hours while the half-life of AZ1₄₈₋₂₂₈ is 3.12±0.84 hours. The results in FIG. 2 show that although the expression of AZ1₄₈₋₂₂₈ in cells decreased with time, AZ1₄₈₋₂₂₈ was degraded more slowly in cells than the AZ1₃₄₋₂₂₈ protein. It can be known from the results in FIG. 3 that the half-life of AZ1₃₄₋₂₂₈ is 1.51±0.30 hours while the half-life of AZ1₆₁₋₂₂₈ is 3.13±0.73 hours; in other words, although the expression of AZ1₆₁₋₂₂₈ in cells decreased with time, AZ1₆₁₋₂₂₈ was degraded more slowly in cells than the AZ1₃₄₋₂₂₈ protein. It can be known from the results in FIG. 4 that the half-life of AZ1₃₄₋₂₂₈ is 1.21±0.21 hours while the half-life of AZ1₇₂₋₂₂₈ is 3.53±1.65 hours; in other words, although the expression of AZ1₇₂₋₂₂₈ in cells decreased with time, AZ1₇₂₋₂₂₈ was degraded more slowly in cells than the AZ1₃₄₋₂₂₈ protein.

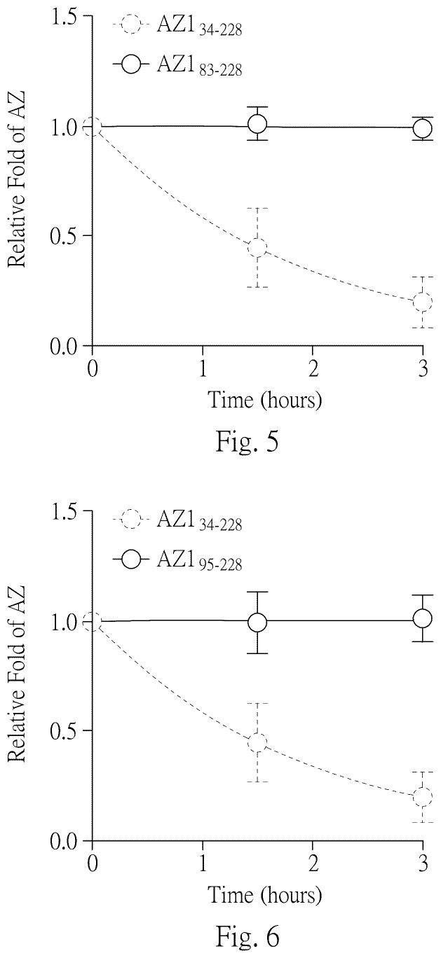

In addition, it can be known from the results in FIG. 5 and FIG. 6 that the half-life of AZ1₃₄₋₂₂₈ is 1.29±0.30 hours in both assays, and that the expression of the AZ1₈₃₋₂₂₈ and AZ1₉₅₋₂₂₈ proteins in cells did not decrease with time. This indicates that the AZ1₈₃₋₂₂₈ and AZ1₉₅₋₂₂₈ proteins had significant anti-degradation ability in a cell environment.

It can be inferred from the results of this example that the degradation of AZ1 proteins in cells is attributable mainly to the 34^{th} to the 71^{st} residues in the N-terminal area.

### Example 4: CHX Chase Assays of AZ Isomers and Variants

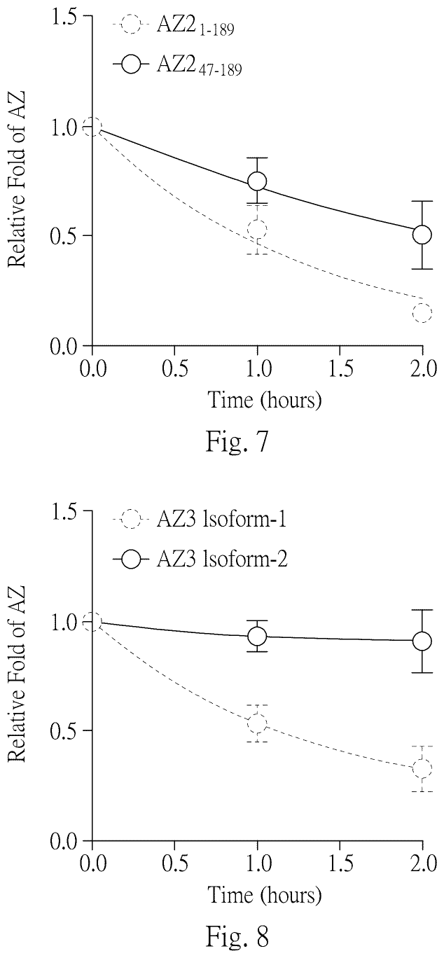

Using the method disclosed in example 3, CHX chase assays were performed on AZ2₁₋₁₈₉, AZ2₄₇₋₁₈₉, AZ3 isoform-1, and AZ3 isoform-2 to find out how these proteins were degraded in cells and the half-lives of the proteins. The results are shown in FIG. 7 and FIG. 8.

It can be known from the results in FIG. 7 that the half-life of AZ2₁₋₁₈₉ is 0.91±0.17 hour while the half-life of AZ2₄₇₋₁₈₉ is 2.12±0.59 hours. This indicates that although the expression of both AZ2₁₋₁₈₉ and AZ2₄₇₋₁₈₉ decreased as the testing time progressed, AZ2₄₇₋₁₈₉ was degraded more slowly than AZ2₁₋₁₈₉. In other words, AZ2₄₇₋₁₈₉ showed a certain degree of resistance to degradation.

Furthermore, it can be known from the results in FIG. 8 that the half-life of AZ3 isoform-1 is 1.09±0.52 hours, and that the expression of AZ3 isoform-2 did not decrease with time. This indicates that only AZ3 isoform-2 showed resistance to degradation in a cell environment. Given that the difference in amino acid sequence between AZ3 isoform-1 and AZ3 isoform-2 is in the N-terminal area, that the length of the N-terminal area of AZ3 isoform-1 is equivalent to that of AZ1 or AZ2, and that the length of the N-terminal area of AZ3 isoform-2 is equivalent to those of the N-terminal truncated AZIs, it can be inferred from the results in FIG. 8 that the length of the N-terminal area (i.e., the number of the N-terminal residues) of an AZ protein does affect how the AZ protein is degraded in cells.

### Example 5: Effects of AZ Variants on ODC Degradation

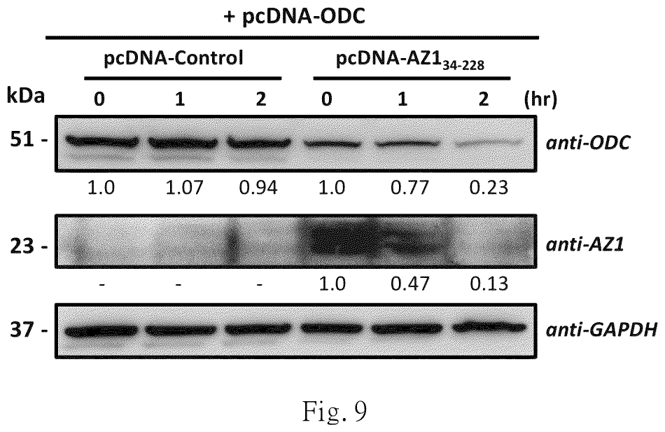

Genes each encoding one of AZ1₃₄₋₂₂₈, AZ1₄₈₋₂₂₈, AZ1₆₁₋₂₂₈, AZ1₇₂₋₂₂₈, AZ1₈₃₋₂₂₈, and AZ1₉₅₋₂₂₈ were separately constructed in plasmid pcDNA3.1 along with the ODC gene (the ratio of ODC to each AZ variant being 9:1), and the resulting plasmids were transfected into HEK293T cells separately, before the cells were cultured for 24 hours. In addition, plasmid pcDNA3.1 containing only the ODC gene was transfected into HEK293T cells, and these cells were cultured for 24 hours as the blank control group. Once the cultivation of each group of HEK293T cells was completed, the expression of ODC and/or the AZ variant in each group of HEK293T cells was tested by the immunoblot method, the concentration and half-life of the AZ protein variant in each group of cells were determined by the CHX chase assay steps disclosed in example 3, and the polyamine content of each group of cells was measured. The results of this example are shown in FIG. 9 to FIG. 19.

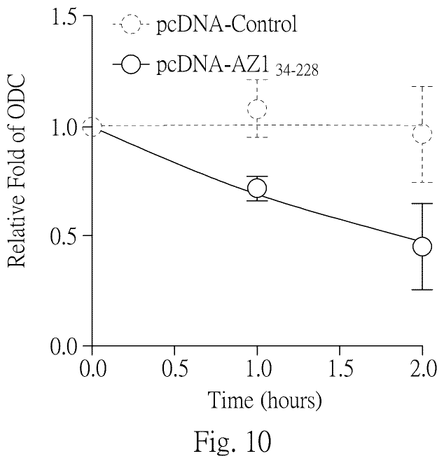

It can be known from the results in FIG. 10 that the half-life of the AZ1₃₄₋₂₂₈ protein is 1.85±0.57 hours. Moreover, the results in FIG. 9 and FIG. 10 show that the ODC protein in the HEK293T cells where the AZ1₃₄₋₂₂₈ protein was expressed was gradually degraded, whereas the ODC in the cell environment without AZ1 expression was not degraded.

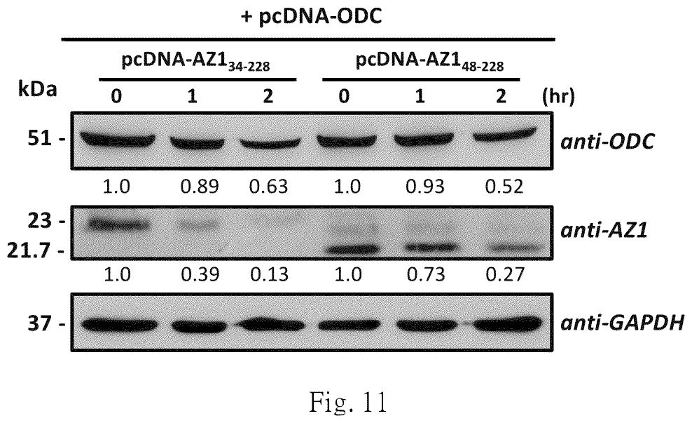

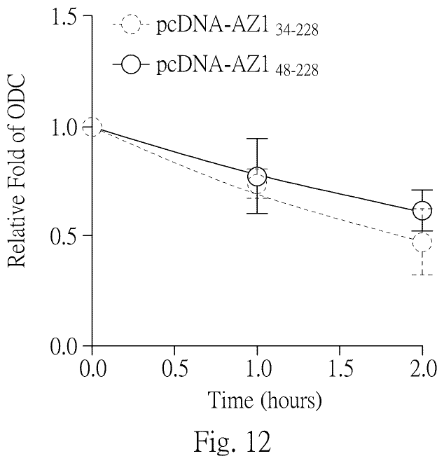

It can be known from the results in FIG. 12 that the half-life of the AZ1₃₄₋₂₂₈ protein is 1.99±0.51 hours, and that the half-life of the AZ1₄₈₋₂₂₈ protein is 2.82±1.02 hours. Moreover, the results in FIG. 11 and FIG. 12 show that the ODC protein in the HEK293T cells where the AZ1₄₈₋₂₂₈ protein was expressed showed a tendency of gradual degradation, and that the presence of the AZ1₄₈₋₂₂₈ protein had a similar effect on ODC degradation to that of the AZ1₃₄₋₂₂₈ protein.

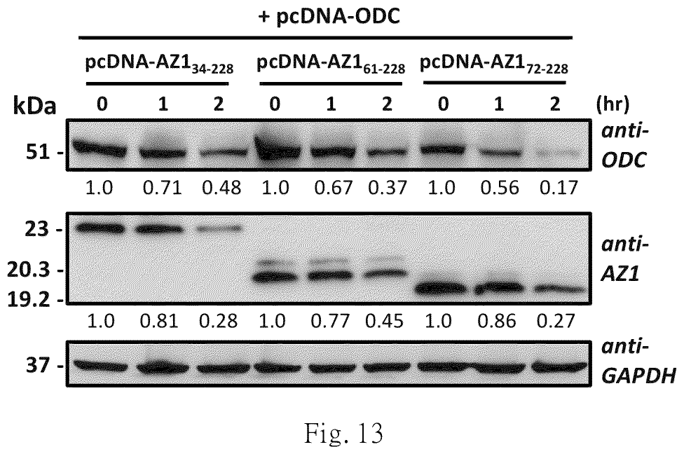

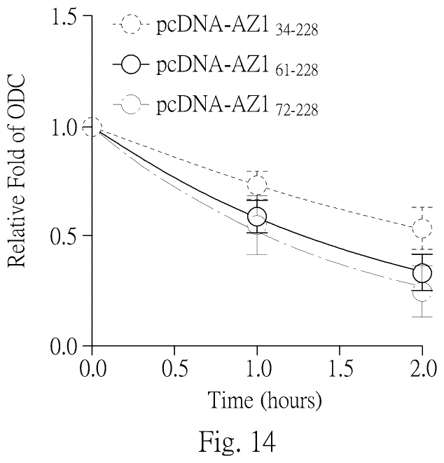

The results in FIG. 13 and FIG. 14 show that the ODC protein was degraded more significantly in HEK293T cells where the AZ1₆₁₋₂₂₈ or AZ1₇₂₋₂₂₈ protein was expressed. It can be further known from the results in FIG. 14 that the AZ1₆₁₋₂₂₈ and AZ1₇₂₋₂₂₈ proteins had a more significant effect on the degradation of the ODC in cells than the AZ1₃₄₋₂₂₈ protein. The half-life of the AZ1₃₄₋₂₂₈ protein is 2.19±0.37 hours, the half-life of the AZ1₆₁₋₂₂₈ protein is 1.28±0.17 hours, and the half-life of the AZ1₇₂₋₂₂₈ protein is 1.06±0.24 hours.

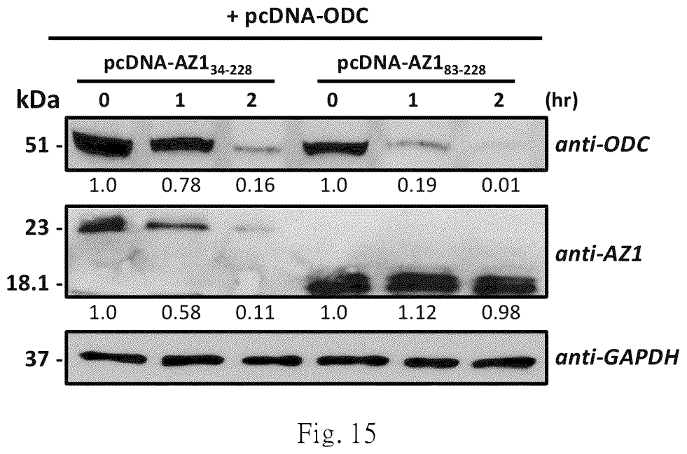

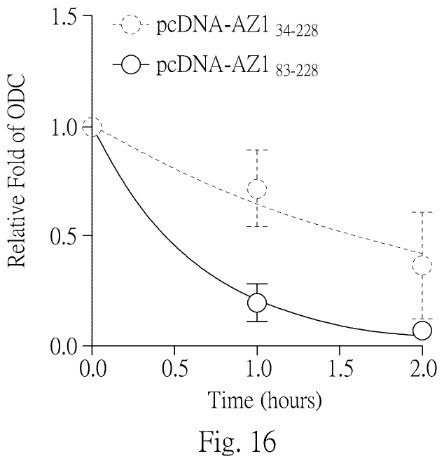

It can be known from the results in FIG. 15 and FIG. 16 that the ODC protein was degraded faster in cells where the AZ1₈₃₋₂₂₈ protein was expressed than in cells where the AZ1₃₄₋₂₂₈ protein was expressed. It can be further known from the results in FIG. 16 that within 1 hour, the amount of the ODC protein remaining in cells where the AZ1₈₃₋₂₂₈ protein was expressed was lower than 25%, whereas the amount of the ODC protein remaining in cells where the AZ1₃₄₋₂₂₈ protein was expressed was higher than 75%. The half-life of the AZ1₃₄₋₂₂₈ protein is 1.60±0.81 hours, and the half-life of the AZ1₈₃₋₂₂₈ protein is 0.44±0.07 hour.

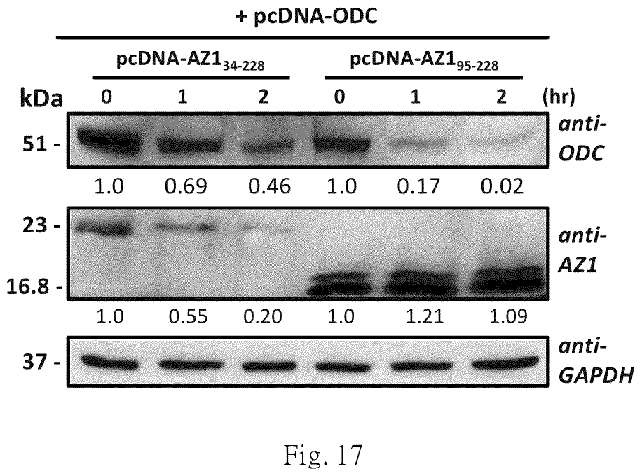

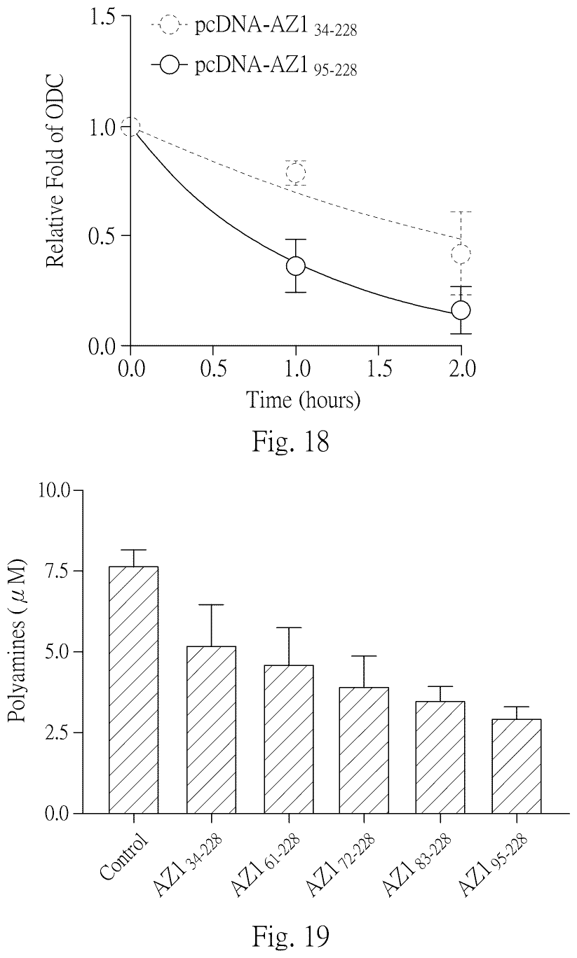

It can be known from the results in FIG. 17 and FIG. 18 that the ODC protein was significantly degraded in cells where the AZ1₉₅₋₂₂₈ protein was expressed. Moreover, compared with the AZ1₈₃₋₂₂₈ protein, the AZ1₉₅₋₂₂₈ protein increased the degradation of the ODC protein in cells, and this indicates that removing the N-terminal area of an AZ1 protein does not increase the ODC degrading ability of the resulting AZ protein variant. It can be further known from the results in FIG. 18 that the half-life of the AZ1₃₄₋₂₂₈ protein is 1.93±0.72 hours while the half-life of the AZ1₉₅₋₂₂₈ protein is 0.72±0.16 hour.

A comparison between FIG. 16 and FIG. 18 reveals that the AZ1₈₃₋₂₂₈ protein and the AZ1₉₅₋₂₂₈ protein were barely degraded at the end of 2 hours. The results in FIG. 9 to FIG. 18 also show that the half-life of the ODC protein in each group of cells was inversely proportional to the stability of the AZ protein in the group of cells, and that the stability of the AZ1₈₃₋₂₂₈ protein and of the AZ1₉₅₋₂₂₈ protein in cells contributed to increasing ODC degradation, meaning the presence of the AZ1₈₃₋₂₂₈ protein and of the AZ1₉₅₋₂₂₈ protein in cells shortens the half-life of the ODC protein.

Moreover, it can be known from the results in FIG. 19 that the expression of polyamines in cells where an N-terminal truncated AZ1 protein (i.e., an AZ variant) was expressed was significantly lower than that in cells lacking an AZ1 gene (pcDNA-control), and that a more significant decrease in polyamine content was found in cells where the AZ1₈₃₋₂₂₈ or AZ1₉₅₋₂₂₈ protein was expressed than in cells where the AZ1₃₄₋₂₂₈ protein was expressed.

It can be inferred from the results in FIG. 1 to FIG. 19 that the AZ1₈₃₋₂₂₈ or AZ1₉₅₋₂₂₈ protein is hardly degraded in cells and can effectively reduce the expression of polyamines in cells. That is to say, administering an effective amount of the AZ1₈₃₋₂₂₈ or AZ1₉₅₋₂₂₈ protein, or a composition containing the AZ1₈₃₋₂₂₈ or AZ1₉₅₋₂₂₈ protein, to an individual can produce a cancer-treating or cancer-preventing effect by regulating the ODC protein expression and/or polyamine content in cells.

### Example 6: Evaluation of the Stability of AZ Protein Variants by Differential Scanning Calorimetry

33 μM AZ variants (AZ1₃₄₋₂₂₈, AZ1₄₈₋₂₂₈, AZ1₆₁₋₂₂₈, AZ1₇₂₋₂₂₈, AZ1₈₃₋₂₂₈, and AZ1₉₅₋₂₂₈) were each dissolved in a solution containing 500 mM sodium chloride (NaCl) and 30 mM Tris-HCl (pH 7.4), and the heat capacity curves of the AZ variants were obtained through a differential scanning calorimeter. The melting-point temperatures (Tₘ1 and Tₘ2) of each AZ protein variant and the enthalpy change (ΔH) of each AZ protein variant during phase change were then derived from the corresponding heat capacity curve, as listed in Table 5.

TABLE 5
Melting-point temperatures (Tₘ1 and Tₘ2) of each AZ variant and
the enthalpy change of each AZ variant during phase change

| | | ΔH1 | | ΔH2 |
|---|---|---|---|---|
| | Tₘ1 | (cal/mol) | Tₘ2 | (cal/mol) |
| | (° C.) | (×10⁵) | (° C.) | (×10⁵) |
| AZ1₃₄₋₂₂₈ | 57.9 ± 0.4 | 3.11 ± 0.16 | 67.6 ± 0.2 | 1.35 ± 0.16 |
| AZ1₄₈₋₂₂₈ | 58.3 ± 0.7 | 2.15 ± 0.17 | 71.7 ± 0.2 | 2.28 ± 0.16 |
| AZ1₆₁₋₂₂₈ | 59.7 ± 0.7 | 3.48 ± 0.22 | 73.6 ± 0.1 | 2.85 ± 0.20 |
| AZ1₇₂₋₂₂₈ | 66.1 ± 1.5 | 2.64 ± 0.56 | 75.3 ± 0.3 | 3.13 ± 0.54 |
| AZ1₈₃₋₂₂₈ | 67.5 ± 0.8 | 2.94 ± 0.34 | 77.4 ± 0.3 | 1.98 ± 0.33 |
| AZ1₉₅₋₂₂₈ | 61.7 ± 0.5 | 1.82 ± 0.11 | 72.6 ± 0.1 | 1.17 ± 0.10 |

It can be known from the results in Table 5 that the two melting-point temperatures of the AZ1₃₄₋₂₂₈ protein are 57.9° C. and 67.6° C., and that each of the other AZ protein variants has two distinctive melting-point temperatures too. AZ1₄₈₋₂₂₈ showed thermal stability at 58.3° C. and 71.7° C., which are about 4° C. higher than the Tₘ2 value of AZ1₃₄₋₂₂₈. AZ1₆₁₋₂₂₈ showed thermal stability at 59.7° C. and 73.6° C., which Tₘ values are about 6° C. higher than the Tₘ2 value of AZ1₃₄₋₂₂₈. AZ1₇₂₋₂₂₈ showed thermal stability at 66.1° C. and 75.3° C., which Tₘ values are about 8° C. and 7.7° C. higher than the Tₘ1 and Tₘ2 values of AZ1₃₄₋₂₂₈ respectively. The Tₘ values of AZ1₈₃₋₂₂₈ are 67.5° C. and 77.4° C., which are about 9.6° C. and 9.8° C. higher than the Tₘ1 and Tₘ2 values of AZ1₃₄₋₂₂₈ respectively. The Tₘ values of AZ1₉₅₋₂₂₈ are 61.7° C. and 72.6° C., which are about 3.8° C. and 5.0° C. higher than the Tₘ1 and Tₘ2 values of AZ1₃₄₋₂₂₈ respectively.

The foregoing results indicate that the AZ1₉₅₋₂₂₈ protein showed relatively high stability among the AZ variants, all of which were relatively unstable in fact.

What is claimed is:

1. A method of preventing and/or treating cancer by a small-molecule protein, comprising: administering an effective amount of the small-molecule protein, or a composition containing the small-molecule protein, to an individual to treat cancer or slow down cancer progression, wherein the small-molecule protein has an amino acid sequence comprising SEQ ID No.: 1, SEQ ID No.: 2, or an amino acid sequence having at least 95% homology with SEQ ID No.: 1 or SEQ ID No.: 2.

2. The method of preventing and/or treating cancer by a small-molecule protein as claimed in claim 1, wherein the amino acid sequence of the small-molecule protein consists of SEQ ID No.: 1 or SEQ ID No.: 2.

3. The method of preventing and/or treating cancer by a small-molecule protein as claimed in claim 1, wherein the small-molecule protein is prepared by cutting off some amino acids in an N-terminal area of an antizyme.

4. The method of preventing and/or treating cancer by a small-molecule protein as claimed in claim 1, wherein the composition is an anticancer drug.

5. The method of preventing and/or treating cancer by a small-molecule protein as claimed in claim 1, wherein the composition is a composition for regulating, and thereby inhibiting expression of, polyamines in cells.

6. The method of preventing and/or treating cancer by a small-molecule protein as claimed in claim 1, wherein the composition is a composition for regulating, and thereby promoting degradation of, ornithine decarboxylase in cells.


SEQUENCE LISTING (13 sequences)

SEQ ID NO: 1 - protein, 147 aa, Homo sapiens
MNSQRDHNLSANLFYSDDRLNVTEELTSNDKTRILNVQSRLTDAKRINWRTVLSGGSLYIEIPGGALPEGSKDSFAVLLEFAEEQLRADHVFICFHKNREDRAALLRTFSFLGFEIVRPGHPLVPKRPDACFMAYTFERESSGEEEE

SEQ ID NO: 2 - protein, 135 aa, Homo sapiens
MFYSDDRLNVTEELTSNDKTRILNVQSRLTDAKRINWRTVLSGGSLYIEIPGGALPEGSKDSFAVLLEFAEEQLRADHVFICFHKNREDRAALLRTFSFLGFEIVRPGHPLVPKRPDACFMAYTFERESSGEEEE

SEQ ID NO: 3 - protein, 228 aa, Homo sapiens
MVKSSLQRILNSHCFAREKEGDKPSATIHASRTMPLLSLHSRGGSSSESSRVSLHCCSNPGPGPRWCSDAPHPPLKIPGGRGNSQRDHNLSANLFYSDDRLNVTEELTSNDKTRILNVQSRLTDAKRINWRTVLSGGSLYIEIPGGALPEGSKDSFAVLLEFAEEQLRADHVFICFHKNREDRAALLRTFSFLGFEIVRPGHPLVPKRPDACFMAYTFERESSGEEEE

SEQ ID NO: 4 - protein, 189 aa, Homo sapiens
MINTQDSSILPLSNCPQLQCCRHIVPGPLWCSDAPHPLSKIPGGRGGGRDPSLSALIYKDEKLTVTQDLPVNDGKPHIVHFQYEVTEVKVSSWDAVLSSQSLFVEIPDGLLADGSKEGLLALLEFAEEKMKVNYVFICFRKGREDRAPLLKTFSFLGFEIVRPGHPCVPSRPDVMFMVYPLDQNLSDED

SEQ ID NO: 5 - protein, 235 aa, Homo sapiens
MPCKRCRPSVYSLSYIKRGKTRNYLYPIWSPYAYYLYCYKYRITLREKMLPRCYKSITYKEEEDLTLQPRSCLQCSESLVGLQEGKSTEQGNHDQLKELYSAGNLTVLATDPLLHQDPVQLDFHFRLTSQTSAHWHGLLCDRRLFLDIPYQALDQGNRESLTATLEYVEEKTNVDSVFVNFQNDRNDRGALLRAFSYMGFEVVRPDHPALPPLDNVIFMVYPLERDVGHLPSEPP

SEQ ID NO: 6 - protein, 190 aa, Homo sapiens
MTVPWRPGKRRITYKEEEDLTLQPRSCLQCSESLVGLQEGKSTEQGNHDQLKELYSAGNLTVLATDPLLHQDPVQLDFHFRLTSQTSAHWHGLLCDRRLFLDIPYQALDQGNRESLTATLEYVEEKTNVDSVFVNFQNDRNDRGALLRAFSYMGFEVVRPDHPALPPLDNVIFMVYPLERDVGHLPSEPP

SEQ ID NO: 7 - DNA, 20 bp, synthetic construct
  source 1..20: mol_type = other DNA
atgccgctcctaagcctgca

SEQ ID NO: 8 - DNA, 20 bp, synthetic construct
  source 1..20: mol_type = other DNA
gagagttccagggtctccct

SEQ ID NO: 9 - DNA, 17 bp, synthetic construct
  source 1..17: mol_type = other DNA
ggtccggggcctcggtg

SEQ ID NO: 10 - DNA, 21 bp, synthetic construct
  source 1..21: mol_type = other DNA
cacccacccctgaagatccca

SEQ ID NO: 11 - DNA, 27 bp, synthetic construct
  source 1..27: mol_type = other DNA
aatagtcagagggatcacaatctttca

SEQ ID NO: 12 - DNA, 24 bp, synthetic construct
  source 1..24: mol_type = other DNA
ttctactccgatgatcggctgaat

SEQ ID NO: 13 - DNA, 20 bp, synthetic construct
  source 1..20: mol_type = other DNA
ggcggcagggatccttctct